(19) United States
(12) Patent Application Publication
(10) Pub. No.: US 2020/0253537 A1
(43) Pub. Date: Aug. 13, 2020

(54) SKIN CONDITION AND RISK EVALUATION METHODOLOGY

(71) Applicant: DEB IP LIMITED, Derbyshire (GB)

(72) Inventors: Paul Blount, Derbyshire (GB); John Hines, Derbyshire (GB); Caroline Fellows, Derbyshire (GB); Chris Lang, Derbyshire (GB); Chris Mann, Derbyshire (GB); Kevin Ormandy, Derbyshire (GB)

(21) Appl. No.: 16/648,027

(22) PCT Filed: Sep. 14, 2018
(86) PCT No.: PCT/GB2018/052626

§ 371 Date: Mar. 17, 2020

(30) Foreign Application Priority Data
Sep. 25, 2017 (GB) 1715450.1

(51) Int. Cl. A61B 5/00; G16H 50/30

(52) U.S. Cl. CPC A61B 5/443; A61B 5/445; A61B 5/442; A61B 2503/20; A61B 5/742; G16H 50/30; A61B 5/7275

(57) ABSTRACT

A method of determining a skin health score for a first subject, the method comprising the steps of assessing the health of the skin of a subject using a plurality of measurement systems, each of the plurality of measurement systems configured to provide a first score characteristic of the health of the skin of the subject as determined by said measurement system, and calculating a second score based on a combination of each of the plurality of first scores, said second score indicative of a skin health score for the first subject.

### FIELD OF INVENTION

The invention relates to a methodology for the assessment of skin condition. In particular the methodology allows for a score to be calculated for a person, groups of persons, or facility to allow for best practice to be identified, and to analysis trends in skin condition over time.

### BACKGROUND TO THE INVENTION

It is known that monitoring of best hygiene practices reduces infection rates in healthcare environments. Such monitoring may be performed in real-time or as a series of snap shots over time where trends can be determined. Such monitoring principles also apply to skin care, where monitoring the correct use skin care produces helps to recognise and prevent skin conditions. This is particular relevant in occupational settings where workers may be exposed to a number of different of products which may affect a workers skin condition. It is known that occupational dermatitis within workers can lead to said workers requiring time off work, and therefore it is desirable to be able to monitor occupational skin health and condition.

Skin disease, and conditions associated with skin health, in particular hand skin health, is arguably the most common occupational disease, with occupational contact dermatitis (CD) accounting for up to 95% of all occupational skin disease; irritant contact dermatitis accounts for the majority of these cases. Contact dermatitis can have serious adverse impact on social and occupational aspects of life, including lost days at work, and threat to employment.

In order to develop a best practice of occupational skin care, it is necessary to assess skin health so as to recognise and monitor a multitude of skin conditions and link their prevalence with skin care practices. This has traditionally been difficult due to a reliance on self-reporting and the associated underreporting of skin health.

To this end, it is known for companies or facilities to perform skin condition audits for individuals in a facility or company. Typically such audits are performed on a voluntary basis, and provide a measure of the skin health of a particular individual. Such audits are largely performed visually and simply quantify the number and severity of apparent symptoms of bad skin, providing a one-off score which is only applicable to that particular subject. Further, current scoring methods tend to yield scores which group the majority of results together due to outlying data, limiting their usefulness in prompting action.

An object of the present invention is to mitigate some of the deficiencies of the prior art mentioned above.

### STATEMENTS OF INVENTION

In accordance with an aspect of the invention, there is provided a method of determining a skin health score for a facility, the method comprising the steps of: for each subject of a plurality of subjects associated with the facility; assessing the health of the skin health of a the subject using a plurality of measurement systems, wherein each of the plurality of measurement systems is configured to provide a first score characteristic of the health of the skin of the subject as determined by said measurement system, and calculating a second score based on a combination of each of the plurality of first scores from the plurality of measurement systems, said second score indicative of a skin health score for the first subject, and for the plurality of subjects; calculating a third score representative of the plurality of subjects based on the second score of each subject, wherein the third score is indicative of a skin health score for the facility.

By calculating a single number that characterises and summarises the combined data from a variety of measurement systems, where each measurement system may record a variety of skin health indicators, the present invention yields a concise and simple scaling output that allows a user to readily monitor their skin health. This allows for the diagnosis of early indications of latent skin conditions, which may take the form of a risk factor. Further, the standardisation of the assessment procedure and the use of set measuring systems produces reliable, accurate and reproducible results that can be validly compared with prior or subsequent scores. As the scores are combined in a consistent manner, operator and user bias can be removed. The score can provide an early warning capability for both individuals, groups of individuals and facilities with their associated populations that their skin health is poor. Accordingly, an appropriate response, or best practice can be identified.

The invention allows for an early indication, and therefore likelihood, of a person, or facility, of suffering from a clinical skin condition such as occupational dermatitis. Such preclinical diagnosis provides an early indication of the user's skin condition and allows for both preventative and remedial action to be taken and best practice identified. Further, combining the individual assessment data of a group of subjects into a single score allows for the skin health of a population to be summarised and tracked more easily.

Optionally, the plurality of subjects is representative of a larger population, further comprising the step of mapping the third score onto the larger population.

Optionally, the larger population is the total work force at a particular work site.

An entire facility can be monitored for changes in skin health that could indicate a systemic skin health risk linked to distinct occupational hazards particular to a given facility. Further, this provides a traceable facility or employer rating useful to monitor the base level skin health of their employees, identify sudden changes, long term trends and monitor the results of any treatments implemented on either particular individuals, groups or entire facilities. Facility-wide score are potentially more useful to employers, as they can identify systematic issues with a particular facility rather than unduly focussing on outlying personal scores.

Optionally, the method further comprises the step of identifying and recording information regarding the subject.

Optionally, the information relates to one or more of: age, location, job, location.

Optionally, the method of any preceding claim further comprising the steps of repeating the assessment and calculation steps over time so as to track changes in at least one of the first, second or third score.

Optionally, the method further comprises the step of recognising a trend in historical measurement data and predicting future values of at least one of the first, second and third score.

Accordingly, this provides an early warning capability for both individuals, groups of individuals and facilities with their associated populations. As such, an appropriate response can be triggered, the particular response depending on the threshold that was exceeded, as well as the particular skin health measurements that contribute to the scores.

Optionally, the method further comprises the step of graphically displaying the second or third score.

Optionally, the skin health score is calculated for a subject's hands.

Other aspects of the invention will be apparent from the appended claim set.

### BRIEF DESCRIPTION OF THE DRAWINGS

Embodiments of the present invention will now be described, by way of example only, with reference to the accompanying drawings, in which:

FIG. 1 is a flow chart of a method in accordance with an embodiment of the invention.

### DETAILED DESCRIPTION OF AN EMBODIMENT

In order to provide a score for the purpose of assessing skin condition to provide a single consistent score for a facility, or company, there is provided the herein described methodology. In use the described methodology is applied to a number of individuals within a facility. Each individual is assigned a score which provides an indication of their skin condition. The scores of multiple individuals can be combined so as to provide a score for a population of users, and even an entire facility in which the tests are being performed. Thus the invention provides an objective measure for a population of users, or subjects, for example a company or facility. As the scores are combined in an objective, consistent, manner the can be compared to previous scores to measure changes over time. The scores may also be compared with other target facilities to identify best practice.

The score thus provides a single, easy to monitor indication of skin health, in particular, hand health, to a user or across a facility. Furthermore, as the score is systematically calculated it may be used to track skin health over time and allow comparisons with a benchmark facility.

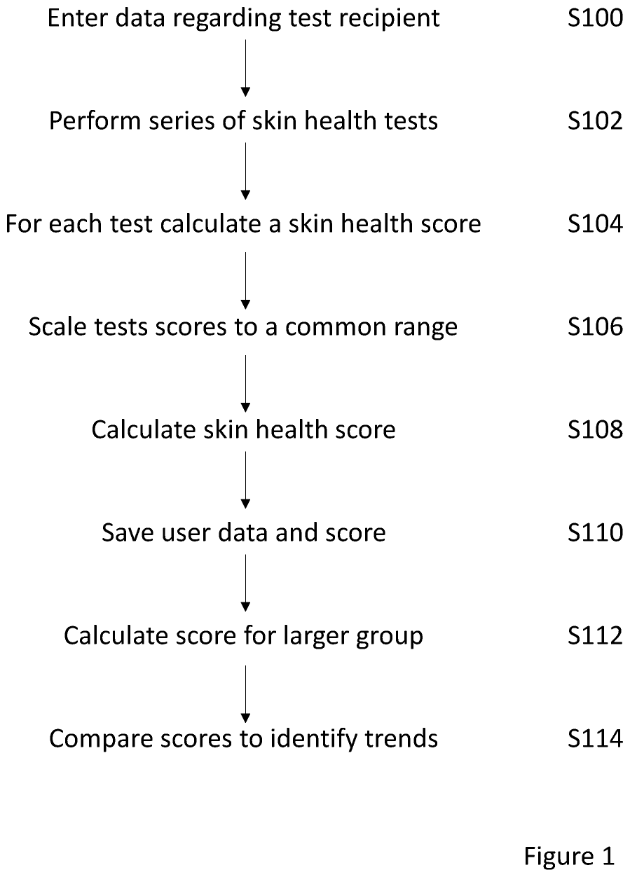

FIG. 1 shows a flow chart of the steps performed to calculate a score representative of users' skin health for a population of users, for example in a facility or company.

In an embodiment the skin health is the hand skin health.

The process commences at step S100. At step 100 a technician (who need not be a medical professional) records data regarding the recipient of the test. For example personal data (age, sex etc.) as well as data regarding the facility and their employment is recorded. In an embodiment the facility is a factory, and the location of the user's work (e.g. factory floor, particular machinery, office etc.) is recorded. This information is used to provide statistical insight into the tests. Such data is recorded onto a computer device such as a desktop computer, laptop, tablet device etc., in a known manner.

At step S102 the technician performs a series of tests to provide different measures of the user's skin health. These tests measure physical attributes of a user's skin, including erythema, scaling, papule density, vesicle density, infiltration, fissuring, surface hydration level, skin barrier function and transepidermal water loss. Other tests may be performed. Preferably, the hand skin health is measured.

The methods by which each attribute is measured are known in the art. For example, a preliminary visual assessment can be carried out covering six morphological signs of skin damage across eight different areas on the hands. The eight areas consist of the front of the fingers, the palms, the back of the hands and back of the fingers on both the left and right hand. If a morphological sign is observed in one out of the eight areas it scores one. If said sign is present in two areas, its scores two, and so on up to a maximum of three. When scoring fissures, a small flat fissure scores 1 and a deep fissure scores 3. Anything in between scores 2. A user can thus score between 0 and 18 in this one particular measurement, with 0 indicating good skin health.

Further measurement can be carried out using one or more of a temperature sensor, humidity sensor, parallel and cross polarised light camera and a chromatic absorption sensor. For example, Erythema is measured using a Mexameter, which uses light reflection to give L-A-B colour values. Five spot measurements are taken on the back of both the left and right hand, The Mexameter than outputs the average Erythema value across both hands, between 0 and 100.

Moisture content is measured using a Corneometer which relies on the capacitance method. A probe is brought into contact with a flat, hairless area of skin. This results in occlusion, as water is accumulated under the probe head, unable to evaporate. Again, five spot measurements are taken on the back of each hand of the user/test subject, yielding a score between 0 and 60.

Transepidermal water loss is measured by means of a Tewameter. The Tewameter operates on the open chamber measuring principle, whereby water evaporates through the chamber and the resulting density gradient is measured by two pairs of sensors. Here, the probe of the Tewameter is held in the area of skin between the thumb and index finger for thirty seconds before yielding a value between 0 and 20, 0 indicating the healthiest skin.

A Visioscope is used to assess scaliness. Scaliness is indicated by the number and thickness of skin flakes collected by a strip of corneofix tape. The tape is pressed on the skin of a user before being held to the Visioscope camera. The Visioscope then produces a value between 0-100% for scaliness.

The results for each test are recorded, either directly from the measurement tool, or inputted manual by the technician.

At step S104, the processor 20 uses the data receives the data from each the test and calculates a first score characterising each physical attribute measured by the measuring tools. Optionally this first score is output to memory 30.

As the scores are measured using different range, (e.g. 0 to 100, 0 to 18 etc.) in an embodiment the scores for each test are scaled at step S106 so that they are all measured along the same range, for example 0 to 10. Such scaling of the scores to the same range may make the calculation of the single score easier.

At step S108, a skin health score for the individual is calculated based on the individual scores. Preferably the skin health score is a measure of the individual's hand skin health score. In further embodiments it is a measure of skin health in general. The skin health score is a single score providing a concise, simple and actionable scaling output indicative of a user's global skin health. In an embodiment, each value outputted by each measurement device or method is normalised as a percentage of the total output, and the resulting values are averaged in order to provide an overall indicator of skin health. In an embodiment where the scores are scaled so as to be measured along the same range a total score may be recorded by summing the scores for each test. In a further embodiments, particular measurement devices or methods are identified as being more useful in the diagnosis of a particular clinical skin condition or pre-condition of interest. In such embodiments these methods are therefore more heavily weighted in the calculation of the user score, which in turn provides measure of user's skin health in relation to a particular condition. Therefore the process may provide a score indicative of overall skin health or a score which focusses more on a particular clinical skin condition.

At step S110 the user skin health score(s) is then saved in the memory along with the data identifying the user. Preferably the score saved at step S110 is a measure of hand health.

Steps S100-S110 are then repeated for a number of distinct users, yielding a set of individual user skin health scores. In an embodiment, these users form a statistically significant sample group of a larger population. In one embodiment, this larger population is the staff of a given institution or a particular facility.

At step S112, the set of user skin health scores are combined into a single score representative of the sample group, and the larger population. Accordingly, for example, two hundred subjects might be measured at a factory with two thousand workers, producing a score applicable to the entire facility. Alternatively users may be subdivided into categories of interest, for example age, job, etc., and scores are assigned for each category. This facility or category score is saved to memory.

As such each user, group of users, or facility may be assigned a score indicative of the skin healthcare.

At step S114, the processor compares presently calculated values of skin health scores (be it user scores, group score or facility scores) with those stored in memory in order to identify trends in every level of the data and avoid skin diseases/injuries by addressing latent risks at an earlier stage. These trends can be used to predict future values of a user, group of facility score. Thus the score provides a monitoring or benchmark score against which future progress and performance may be easily monitored. Furthermore by defining the skin health into a single score which takes into account a number of different tests, it can be presented in an easier to understand manner.

In some embodiments if a predicted score exceeds a predetermined threshold, the system can trigger an output, for example via display 60 or transmission means so as to prompt preventative action at the individual or facility wide level as required. In an embodiment, the scores outputted by the system 10 are transmitted to a remote server and displayed via an internet assessable dashboard, thereby enabling administrators to observe, assess and address the scores received by individual staff member and facilities remotely. This further facilitates the comparison and benchmarking of multiple subjects and facilities, which can be tracked and managed over time.

Accordingly the described methodology allows for an automated and systematic collection of the data required to measure skin health. By performing the assessment utilising the system described, operator bias is substantially reduced as a weighted score is calculated in a systematic manner using equipment that produces accurate and reliable data. This further enables collected data to be validly compared with prior and subsequent date collected using the same system and equipment, improving the ability to recognise historical and forecasted trends.

A further advantage is that the system allows for the large scale collation of data. Whilst individual users are assigned a skin health score, the score can be applied to a group of users within a facility or the entire facility itself. For example in a factory setting it may be useful to identify users according to their job or work environment, as office based workers may be less likely to suffer from certain conditions than workers on the factory floor.

Furthermore as the scores are generated and saved they can be viewed and visualised to identify trends over time, or by comparing different facilities, on both a user-by-user and workforce-wide basis. Furthermore, such visualisation may allow a skincare professional to identify best practice and monitor the impact of said best practice over time.

Advantageously scores can be compared to previously recorded scores stored in the memory 30. Thus trends for users, groups of users and facilities can be easily visualised over time. Furthermore, as the scores are weighted and calculated in a consistent manner, scores between different users, groups of users and facilities may be compared allowing for comparison between a predetermined target group and the measured group in order to determine and identify best practice.

1. A method of determining a skin health score for a facility, the method comprising the steps of:
for each subject of a plurality of subjects associated with the facility;
assessing the skin health of the subject using a plurality of measurement systems, wherein each of the measurement systems is configured to provide a first score characteristic of the health of the skin of the subject as determined by said measurement system,
calculating a second score based on a combination of each of the first scores from the plurality of measurement systems, said second score indicative of a skin health score for the subject, and
for the plurality of subjects;
calculating a third score representative of the plurality of subjects based on the second score of each subject, wherein the third score is indicative of a skin health score for the facility.

2. The method of claim 1 wherein the measurement systems measure at least two of erythema, scaling, papule density, vesicle density, infiltration, fissuring, surface hydration level, skin barrier function and transepidermal water loss.

3. The method of claim 1 wherein the plurality of subjects is representative of a larger population, further comprising the step of mapping the third score onto the larger population.

4. The method of claim 3 wherein the larger population is the total work force at a particular work site.

5. The method of claim 1 further comprising the step of identifying and recording information regarding the subject.

6. The method of claim 5 wherein the information relates to one or more of: age, location, job, location.

7. The method of claim 1 further comprising the steps of repeating the assessment and calculation steps over time so as to track changes in at least one of the first, second or third score.

8. The method of claim 7 further comprising the step of recognising a trend in historical measurement data and predicting future values of at least one of the first, second and third score.

9. The method of claim 1 further comprising the step of graphically displaying the second or third score.

10. The method of claim 1 wherein the skin health score is calculated for a subject's hands.
